(19) United States
(12) Patent Application Publication
(10) Pub. No.: US 2023/0021410 A1
(43) Pub. Date: Jan. 26, 2023

(54) Alkyl-Demethylation Processes and Catalyst Compositions Therefor

(71) Applicant: ExxonMobil Chemical Patents Inc., Baytown, TX (US)

(72) Inventors: Umar Aslam, Houston, TX (US); Meha Rungta, Houston, TX (US); Chuansheng Bai, Phillipsburg, NJ (US); Ali A. Kheir, Houston, TX (US); Paul Podsiadlo, Spring, TX (US)

(21) Appl. No.: 17/781,086

(22) PCT Filed: Nov. 16, 2020
(86) PCT No.: PCT/US2020/060682

§ 371 Date: May 31, 2022

Related U.S. Application Data
(60) Provisional application No. 62/950,395, filed on Dec. 19, 2019.

(30) Foreign Application Priority Data
Apr. 21, 2020 (EP) 20170577.9

(51) Int. Cl. B01J 23/46; B01J 23/00; B01J 23/02; B01J 21/08; B01J 35/10; B01J 37/02; C07C 4/18

(52) U.S. Cl. CPC B01J 23/464; B01J 23/005; B01J 23/02; B01J 21/08; B01J 35/1014; B01J 37/0215; C07C 4/18; C07C 2523/46; C07C 2521/10; C07C 2521/04; C07C 2521/08

(57) ABSTRACT

Catalyst compositions to perform selective alkyl-demethylation of C2+-hydrocarbyl-substituted aromatic hydrocarbon may exhibit a hydrogen chemisorption of at least 15% and comprise an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and combinations and mixtures of two or more thereof; and a transition metal element dispersed upon the oxide support material. Alkyl-demethylation processes of a C6+ aromatic hydrocarbon-containing stream comprising C2+-hydrocarbyl-substituted aromatic hydrocarbons may comprise contacting the catalyst compositions in an alkyl-demethylation zone under alkyl-demethylation conditions to form an alkyl-demethylated aromatic hydrocarbon as an effluent exiting the alkyl-demethylation zone.

### CROSS-REFERENCE OF RELATED APPLICATIONS

This application claims priority to and the benefit of U.S. Provisional Application No. 62/950,395, filed Dec. 19, 2019 and EP Application No. 20170577.9, filed Apr. 21, 2020, the disclosures of both of which are incorporated herein by reference in their entirety.

### FIELD

The present disclosure relates to processes and catalyst compositions having good selectivity for converting C2+-substituted aromatic hydrocarbons into C1-substituted aromatic hydrocarbons. Such processes and catalyst compositions may be effective for producing, for example, xylenes, trimethylbenzenes, and the like from heavier aromatic compounds bearing a C2+-alkyl substitute and/or an aliphatic ring annelated to an aromatic ring of the aromatic compound.

### BACKGROUND

Large quantities of p-xylene and o-xylene are consumed worldwide every year. Para-xylene (“p-xylene”) is an important industrial commodity chemical for making terephthalic acid, which is used for making large quantities of polyester fibers. Ortho-xylene (“o-xylene”) is another important industrial commodity chemical for making phthalic acid, which may be used for making plasticizers and other industrial materials. In addition, xylene isomers represent a high-value fuel component in comparison to heavier aromatic compounds, such as those bearing a C2+-alkyl substitute and/or an aliphatic ring annelated to an aromatic ring.

The high demand for xylene isomers has led to the advancement of many technologies for their large-scale fabrication. o-Xylene and p-xylene are often present in C8 aromatic hydrocarbon mixtures additionally comprising meta-xylene and ethylbenzene at various quantities. Separation of p-xylene from such C8 aromatic hydrocarbon mixtures can often be realized through crystallization and adsorption chromatography technologies, for example. The residual filtrate from crystallization-based separation and the raffinate from the adsorption chromatography-based separation (collectively the “raffinate”) are depleted (lean) in p-xylene and rich in m-xylene and o-xylene. In conventional processes, the raffinate may then be isomerized by using an isomerization catalyst to convert a portion of the o- and m-xylenes into p-xylene in a xylenes loop, from which additional p-xylene may be separated.

In a petrochemical plant, a major source of C8 aromatic hydrocarbons is a C6+ hydrocarbon reformate stream produced from a heavy naphtha reforming reactor (“reformer”). In the presence of a reforming catalyst under reforming conditions, paraffins and aromatic hydrocarbons contained in a heavy naphtha feed undergo various complex chemical reactions, such as isomerization, dehydrogenation, dehydrocyclization, aromatization, and the like, to yield a reforming mixture comprising additional branched paraffins, aromatic hydrocarbons, and hydrogen. A C6+ hydrocarbon reformate stream separated from the reforming mixture may comprise benzene, toluene, C8 aromatics, and C9+ aromatics. The C8 aromatics of the C6+ hydrocarbon reformate stream typically comprise, in addition to the xylenes, ethylbenzene at a substantial quantity. The C9+ aromatics of the C6+ hydrocarbon reformate stream typically comprise, in addition to aromatic hydrocarbons comprising only methyl substitutes attached to the aromatic ring therein (e.g., trimethylbenzenes and tetramethylbenzenes), aromatic hydrocarbons comprising at least one C2+ alkyl group (e.g., ethylmethylbenzenes, diethylbenzenes, C3-alkylbenzenes, and the like) and/or aromatic hydrocarbons comprising an aliphatic ring annelated to the aromatic ring (e.g., indane, methylindanes, tetralin, methyltetralins, and the like).

As a result of the foregoing, ethylbenzene and C9+ aromatic hydrocarbons are often present in significant quantities in raffinate and similar hydrocarbon sources, such as steam-cracked naphtha (SCN). SCN streams are oftentimes not considered to be economically viable source materials for producing xylenes due to the high concentrations of ethylbenzene and indane therein. Even in the case of raffinate, strategies for processing the ethylbenzene and indane are usually employed to make this xylenes source material more economically viable and to mitigate other issues during xylenes production. Direct conversion of ethylbenzene and C9+ aromatic hydrocarbons into xylenes via isomerization is often impractical, and a more common strategy to prevent ethylbenzene accumulation in the xylenes loop is to conduct the raffinate isomerization under vapor-phase conditions in the presence of an additional catalyst effective to de-ethylate ethylbenzene to form benzene. Vapor-phase isomerization of this type is energy intensive, however, and the ethylbenzene is not converted into value products, such as xylenes.

To increase the production of xylenes, the C9+ aromatic hydrocarbons in the C6+ hydrocarbon reformate stream can be separated and undergo transalkylation with benzene and/or toluene. In the presence of a suitable transalkylation catalyst under transalkylation conditions, the C9+ aromatic hydrocarbons may exchange methyl groups with benzene and/or toluene to produce additional xylenes from the original source material. Transalkylation usually occurs in under vapor-phase conditions and is likewise rather energy intensive. De-alkylation of at least some of the C9+ aromatic hydrocarbons may occur under the vapor-phase conditions in the presence of a suitable dealkylation catalyst to produce additional benzene and/or toluene in situ during transalkylation, if desired.

As such, there remains a need for more efficient processing of alkylated aromatic hydrocarbons, such as those bearing a C2+-alkyl substitute and/or an aliphatic ring annelated to an aromatic ring, into xylenes and similar value aromatic products bearing methyl substitution upon the aromatic ring. In particular, there remains a need for direct conversion of C8 aromatic hydrocarbons (ethylbenzene) and C9+ aromatic hydrocarbons into aromatic compounds bearing only methyl substitutes, such as xylenes, or a substantial majority of methyl substitutes. Heretofore, direct partial dealkylation of aromatic hydrocarbons bearing one or more alkyl substitutes, particularly ethyl substitutes, larger alkyl substitutes and annelated aliphatic rings, often results in excessive amounts of complete dealkylation and/or aromatic ring loss through hydrogenation and/or ring rupture, thereby resulting in ineffective utilization of the initial hydrocarbon feed. The present disclosure rectifies this existing deficiency and provides other advantages as well.

### SUMMARY

Alkyl-demethylation processes and catalyst compositions effective therefore can be used to convert C2+-hydrocarbyl-substituted aromatic hydrocarbons with high selectivity into alkyl-demethylated aromatic hydrocarbons, particularly methylated aromatic hydrocarbons. The alkyl-demethylation processes and catalyst compositions disclosed herein may afford one or more advantages over conventional techniques for processing aromatic hydrocarbon streams such as (i) improved energy efficiency; (ii) increased production of value aromatic products, such as xylenes; (iii) improved utilization of an aromatic hydrocarbon feed; and (iv) simplified process logistics and equipment.

In a first aspect, the present disclosure provides catalyst compositions for selective alkyl-demethylation of a C2+-hydrocarbyl-substituted aromatic hydrocarbon. The catalyst compositions comprise: an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and combinations and mixtures of two or more thereof; and a transition metal element dispersed upon the oxide support material; wherein the catalyst compositions exhibit a hydrogen chemisorption value of at least 15%.

In a second aspect, the present disclosure provides processes for converting a C2+-hydrocarbyl-substituted aromatic hydrocarbon into methylated aromatic hydrocarbons. The processes comprise: providing a C6+aromatic hydrocarbon-containing stream comprising the C2+-hydrocarbyl-substituted aromatic hydrocarbon, wherein the C2+-hydrocarbyl-substituted aromatic hydrocarbon has (i) a C2+ alkyl substitute attached to an aromatic ring therein and/or (ii) an aliphatic ring annelated to an aromatic ring therein; and contacting the C6+ aromatic hydrocarbon-containing stream with the catalyst composition of the first aspect in an alkyl-demethylation zone under alkyl-demethylation conditions effective to convert at least a portion of the C2+-hydrocarbyl-substituted aromatic hydrocarbon to an alkyl-demethylated aromatic hydrocarbon comprising at least one methyl substitute to obtain a first alkyl-demethylated effluent exiting the alkyl-demethylation zone.

In a third aspect, processes for making catalyst compositions for selective alkyl-demethylation of a C2+-hydrocarbyl-substituted aromatic hydrocarbon are disclosed herein. The processes comprise: providing an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and mixtures and combinations of two or more thereof; providing a source material of a transition metal element; dispersing the source material of the transition metal element on the oxide support material to obtain a catalyst composition precursor; and contacting the catalyst composition precursor with a reducing atmosphere under activating conditions to obtain the catalyst composition. Catalyst compositions prepared according to the third aspect, catalyst composition precursors prepared according to the third aspect, and catalytic processes using catalyst compositions and catalyst composition precursors prepared according to the third aspect are also provided herein.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a graph showing illustrative conversion performance for 0.5% Rh on a spinel formed from calcined hydrotalcite in comparison to 0.5% Rh on alumina for catalytic treatment of xylenes/ethylbenzene.

FIG. 2 is a graph showing illustrative conversion performance for 1.0% Rh on a spinel formed from calcined hydrotalcite in comparison to 1.0% Rh on alumina for catalytic treatment of a HAR feed.

### DETAILED DESCRIPTION

The present disclosure relates to conversion of aromatic hydrocarbons bearing a C2+-alkyl substitute and/or an aliphatic ring annelated to an aromatic ring into methyl-substituted aromatic hydrocarbons and, more specifically, catalytic demethylation of such aromatic hydrocarbons using catalyst compositions having minimal propensity to promote complete dealkylation and/or aromatic ring loss.

Various specific embodiments, versions and examples of this disclosure will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process may be described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite articles “a” or “an” shall mean “at least one” unless specified to the contrary or the context clearly indicates otherwise. Thus, for example, embodiments using “a fractionation column” include embodiments where one, two or more fractionation columns are used, unless specified to the contrary or the context clearly indicates that only one fractionation column is used.

“Consisting essentially of,” as used herein, means a composition, feed, or effluent includes a given component or group of components at a concentration of at least 60 wt %, preferably at least 70 wt %, more preferably at least 80 wt %, more preferably at least 90 wt %, or still more preferably at least 95 wt %, based on the total weight of the composition, feed, or effluent.

“Hydrocarbon” means (i) any compound consisting of hydrogen and carbon atoms or (ii) any mixture of two or more such compounds in (i). The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of them at any proportion. A “Cm to Cn hydrocarbon” or “Cm-Cn hydrocarbon,” where m and n are positive integers and m<n, means any of Cm, Cm+1, Cm+2, . . . , Cn−1, Cn hydrocarbons, or any mixtures of two or more thereof. Thus, a “C2 to C3 hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn− hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

“Light hydrocarbon” means any C5-hydrocarbon.

As used herein, “wt %” means percentage by weight, “vol %” means percentage by volume, “mol %” means percentage by mole, “ppm” means parts per million, and “ppm wt” and “wppm” are used interchangeably to mean parts per million on a weight basis. All concentrations herein are expressed on the basis of the total amount of the composition in question. All ranges expressed herein should include both end points as two specific embodiments unless specified or indicated to the contrary.

Nomenclature of elements and groups thereof used herein are pursuant to the Periodic Table used by the International Union of Pure and Applied Chemistry after 1988. An example of the Periodic Table is shown in the inner page of the front cover of Advanced Inorganic Chemistry, 6^{th} Edition, by F. Albert Cotton et al. (John Wiley & Sons, Inc., 1999).

“Liquid-phase” means reaction conditions in which aromatic hydrocarbons present in a reactor are substantially in liquid phase. “Substantially in liquid phase” means ≥90 wt %, preferably ≥95 wt %, preferably ≥99 wt %, and preferably the entirety of the aromatic hydrocarbons, is in liquid phase.

“Vapor-phase” means reaction conditions in which aromatic hydrocarbons present in a reactor are substantially in vapor phase. “Substantially in vapor phase” means ≥90 wt %, preferably ≥95 wt %, preferably ≥99 wt %, and preferably the entirety of the aromatic hydrocarbons, is in vapor phase.

“Methylated aromatic hydrocarbon” means an aromatic hydrocarbon comprising at least one methyl group and only methyl group(s) attached to the aromatic ring(s) therein. Examples of methylated aromatic hydrocarbons include toluene, xylenes, trimethylbenzenes, tetramethylbenzenes, pentamethylbenzene, hexamethylbenzene, methylnaphthalenes, dimethylnaphthalenes, trimethylnaphthalenes, tetramethylnaphthalenes, and the like.

“C2+-hydrocarbyl-substituted aromatic hydrocarbon” means an aromatic hydrocarbon comprising a substituted aromatic ring bearing a hydrocarbyl group, other than a methylated aromatic hydrocarbon bearing only methyl groups. A C2+-hydrocarbyl-substituted aromatic hydrocarbon may comprise (i) a C2+-hydrocarbyl group (e.g., a C2+-alkyl group) attached to an aromatic ring therein and/or (ii) an aliphatic ring annelated to an aromatic ring therein. Examples of C2+-hydrocarbyl-substituted aromatic hydrocarbons in scenario (i) include, but are not limited to (carbon numbers in parentheses): ethylbenzene (C8); ethylmethylbenzenes (C9); n-propylbenzene (C9); cumene (C9); ethyldimethylbenzenes (C10); diethylbenzenes (C10); n-propylmethylbenzenes (C10); methylcumenes (i.e., isopropylmethylbenzenes, C10); n-butylbenzene (C10); sec -butylbenzene (C10); tert-butylbenzene (C10); and the like. Examples of C2+-hydrocarbyl-substituted aromatic hydrocarbons in scenario (ii) include, but are not limited to (carbon numbers in parentheses): indane (C9); indene (C9); methylindanes (C10); methylindenes (C10); tetralin (C10); methyltetralin (C11), dimethylindanes (C11); ethylindanes (C11); and the like. Benzene and naphthalene are neither methylated aromatic hydrocarbons nor C2+-hydrocarbyl-substituted aromatic hydrocarbons.

“Alkyl-demethylation” means, in the presence of a suitable catalyst and molecular hydrogen, (i) the removal of one or more carbon atoms from a Cm (m≥2) alkyl group attached to an aromatic ring to leave a Cm' residual alkyl group attached to the aromatic ring, wherein 1≤m′≤m−1, preferably m′=1; or (ii) the removal of one or more carbon atoms from a Cn aliphatic ring annelated to an aromatic ring to leave one or more residual alkyl groups (preferably methyl) comprising n′ carbon atoms in total attached to an aromatic ring, wherein 1≤n′≤n−1. Reactions (i) and (ii) are collectively called “alkyl-demethylation reactions” in this disclosure. Thus, alkyl-demethylation of a C2+-hydrocarbyl-substituted aromatic hydrocarbon comprising a Cm (m≥2) alkyl group attached to an aromatic ring therein can result in an aromatic hydrocarbon substituted by a Cm-1 alkyl group, or a Cm-2 alkyl group, . . . , or a methyl group, as an alkyl-demethylated hydrocarbon. Alkyl-demethylation of a C2+-hydrocarbyl-substituted aromatic hydrocarbon comprising an n-member (n≥5) aliphatic ring annelated to an aromatic ring therein can result in aromatic hydrocarbons substituted by at least one substitute (preferably two methyls) taken together having n-1, n-2, n-3, n-4, . . . , or 1 carbon atoms. The removed methyl group(s) forms light hydrocarbon(s) (preferably methane) in the presence of molecular hydrogen. With respect to C2+-hydrocarbyl-substituted aromatic hydrocarbons, the catalyst is desirably selective toward alkyl-demethylation defined above over (i) the removal of the Cn (n≥2) group attached to an aromatic ring in its entirety leaving no residual substitute and (ii) the removal of a methyl group attached to an aromatic ring leaving no residual substitute. Thus, further alkyl-demethylation of the alkyl-demethylated hydrocarbon(s) which are also C2+-hydrocarbyl-substituted aromatic hydrocarbons can result in increased amount of methylated aromatic hydrocarbons (e.g., tetramethylbenzenes, trimethylbenzenes, xylenes, and toluene). Without intending to be bound by a particular theory, such methylated aromatic hydrocarbons can be produced from C2+-hydrocarbyl-substituted aromatic hydrocarbons with or without the formation of the alkyl-demethylated hydrocarbons as intermediate C2+-hydrocarbyl-substituted aromatic hydrocarbons. Desirably, treating an aromatic hydrocarbon feed mixture comprising C2+-hydrocarbyl-substituted aromatic hydrocarbons by alkyl-demethylation may produce an aromatic hydrocarbon product mixture having a higher methyl to aromatic ring molar ratio compared to the feed mixture. Examples of alkyl-demethylation reactions of C2+-hydrocarbyl-substituted aromatic hydrocarbons to produce alkyl-demethylated hydrocarbon(s) include, but are not limited to the following (products marked with an asterisk (*) can be formed through hydrogenolysis of an annelated aliphatic ring without loss of one or more carbon atoms; these products may undergo subsequent alkyl-demethylation according to the disclosure herein):

| C2+-hydrocarbyl-substituted | Alkyl-demethylated hydrocarbon |
|---|---|
| aromatic hydrocarbon | product(s) |
| Ethylbenzene | Toluene |
| n-Propylbenzene | Ethylbenzene; toluene |
| Cumene | Ethylbenzene; toluene |
| Ethylmethylbenzenes | Xylenes |
| Indane | Ethylmethylbenzenes(*); |
| | n-propylbenzene(*); cumene(*); |
| | ethylbenzene; xylenes; toluene |
| 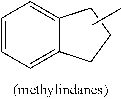 | Indane; propylmethylbenzenes(*); diethylbenzenes(*); ethylmethylbenzenes; butylbenzenes(*); n-propylbenzene; cumene; ethylbenzene; xylenes; toluene |
| 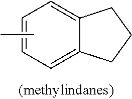 | Ethylmethylbenzenes; ethyldimethylbenzene(*); trimethylbenzenes; butylmethylbenezenes(*); propylmethylbenzenes(*); xylenes |
| 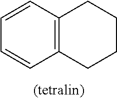 | Propylmethylbenzenes(*); diethylbenzene(*), butylbenzenes(*); n-propylbenzene; cumene; ethylmethylbenzenes; xylenes; ethylbenzene; toluene |

“Dealkylation” of an alkyl group attached to an aromatic ring means the removal of the alkyl group in its entirety leaving no residual group attached to the aromatic ring. Thus, demethylation of the methyl group in toluene to form benzene, deethylation of the ethyl group in ethylbenzene to form benzene, deethylation of the ethyl group in ethylmethylbenzenes to form toluene, and the depropylation of the isopropyl group in cumene to form benzene are a specific forms of dealkylation. Dealkylation of an alkylated aromatic hydrocarbon is typically effected in the presence of a catalyst selective for dealkylation over alkyl-demethylation, discussed above, in the presence of molecular hydrogen. The removed alkyl group in the dealkylation reaction forms light hydrocarbon(s) in the presence of molecular hydrogen.

An effluent or a feed is sometimes also called a stream in this disclosure. Where two or more streams are shown to form a joint stream and then supplied into a vessel, it should be interpreted to include alternatives where the streams are supplied separately to the vessel where appropriate. Likewise, where two or more streams are supplied separately to a vessel, it should be interpreted to include alternatives where the streams are combined before entering into the vessel as joint stream(s) where appropriate.

### The Alkyl-Demethylation Processes of This Disclosure

Alkyl-demethylation processes of the present disclosure may occur in the presence of a suitable catalyst under a set of alkyl-demethylation conditions in an alkyl-demethylation zone of a reactor. On contacting a suitable catalyst, which may be formed from a precursor thereof, under the alkyl-demethylation conditions, a Cm+(m≥2) alkyl group attached to an aromatic ring (e.g., a benzene ring, a naphthalene ring, and the like) loses one or more distal carbon atoms (i.e., a carbon atom from the alkyl group attached to the aromatic ring) to form preferably a methylated aromatic hydrocarbon with a methyl group attached to the aromatic ring. Preferably, the catalyst favors alkyl-demethylation of a Cm (m≥2) alkyl group attached to an aromatic ring over the demethylation of a methyl group attached to an aromatic ring under the alkyl-demethylation conditions. Thus, alkyl-demethylation of ethylbenzene (i.e., ethyl-demethylation) in the presence of a catalyst having suitable alkyl-demethylation specificity may result in the net production of toluene, with further demethylation to produce benzene being disfavored or occurring to a lesser extent. Alkyl-demethylation of ethylmethylbenzenes in the presence of a catalyst having suitable alkyl-demethylation specificity results in the net production of xylenes, with further demethylation to produce toluene and benzene being disfavored or occurring to a lesser extent. Similarly, alkyl-demethylation of C3-alkylbenzenes (i.e., benzene substituted by a single C3-alkyl group) in the presence of a suitable catalyst preferably produces toluene. Alkyl-demethylation of C3-alkylmethylbenzenes preferably results in the net production of xylenes. Thus, alkyl-demethylation processes may favor the production of methylated aromatic hydrocarbons (toluene, xylenes, trimethylbenzenes, and the like) over the production of benzene in the presence of a catalyst having suitable alkyl-demethylation specificity. Processes for converting aromatic hydrocarbons according to this disclosure can advantageously comprise one or more alkyl-demethylation process steps.

In the presence of a suitable catalyst under alkyl-demethylation conditions, aromatic hydrocarbons comprising an aliphatic ring annelated to an aromatic ring (e.g., indane, methylindanes, tetralin, methyltetralins, and the like) likewise may undergo scission of the aliphatic ring to form one or more linear or branched residual groups attached to the aromatic ring, with or without first losing a carbon atom from the aliphatic ring. Any C2+ linear or branched residual alkyl group formed via ring scission may subsequently undergo one or more steps of alkyl-demethylation reactions to be eventually converted into a methyl group attached to the aromatic ring, the further demethylation of which is disfavored in the presence of a catalyst having suitable specificity, as discussed herein. Thus, C2+-hydrocarbyl-substituted aromatic hydrocarbons such as indane, methylindanes, tetralin, and methyltetralins can be converted into methylated aromatic hydrocarbons in the alkyl-demethylation processes of the present disclosure. In the processes of the present disclosure including an alkyl-demethylation step occurring in an alkyl-demethylation zone, preferably the catalyst is capable of catalyzing the scission of aliphatic ring(s) annelated to an aromatic ring, with alkyl-demethylation taking place following aliphatic ring scission. In case the catalyst capable of promoting alkyl-demethylation is not sufficiently active for catalyzing scission of the aliphatic ring(s), an additional catalyst capable of promoting aliphatic ring scission, preferably in a selective manner, may be included in the alkyl-demethylation zone as well.

While alkyl-demethylation reactions as described above are favored in the alkyl-demethylation processes of this disclosure, it should be understood that certain side reactions other than the alkyl-demethylation reactions may occur to some degree in the presence of the catalyst under the alkyl-demethylation reaction conditions present in the alkyl-demethylation zone of the reactor.

The hydrocarbon feed supplied to an alkyl-demethylation zone in the processes of this disclosure may comprise a C6+ aromatic hydrocarbon-containing stream comprising one or more C2+-hydrocarbyl-substituted aromatic hydrocarbons. The concentration of the C2+-hydrocarbyl-substituted aromatic hydrocarbons in the hydrocarbon feed can range from c1 to c2 wt %, based on the total weight of the C6+ aromatic hydrocarbons in the feed within the zone, wherein c1 and c2 can be, independently, e.g., 2, 4, 5, 6, 8, 10, 15, 20, 25, 30, 35, 40, 45, 50, 55, 60, 65, 70, 75, 80, 85, 90, as long as c1<c2. Thus, the feed subject to alkyl-demethylation according to the disclosure herein can comprise such C2+-hydrocarbyl-substituted aromatic hydrocarbons at relatively low to very high concentrations, depending on the source of the feed.

In certain embodiments, the hydrocarbon feed supplied to the alkyl-demethylation zone can comprise C8 aromatics including ethylbenzene and xylenes at various concentrations. In certain embodiments, the concentration of ethylbenzene in the feed supplied to the alkyl-demethylation zone can range from c(EB)1 to c(EB)2 wt %, based on the total weight of the C8 aromatic hydrocarbons contained in the feed, wherein c(EB)1 and c(EB)2 can be, independently, e.g., 2, 4, 5, 6, 8, 10, 15, 20, 25, 30, 35, 40, 45, or 50, as long as c(EB)1<c(EB)2. The processes of this disclosure can be particularly advantageous when used to process such streams comprising high concentrations of ethylbenzene such as at ≥10 wt %, ≥20 wt %, or ≥30 wt %, based on the weight of all C8 aromatic hydrocarbons in the feed, to produce toluene. Advantageously, processing the ethylbenzene in this manner can minimize accumulation of the ethylbenzene in a xylenes loop. Through processes known to those having ordinary skill in the art, the toluene can subsequently be converted into additional quantities of xylenes, particularly p-xylene, via methylation with methanol and/or dimethyl ether, toluene disproportionation, and/or transalkylation with C9+aromatic hydrocarbons, particularly methylated aromatic hydrocarbons such as trimethylbenzenes and tetramethylbenzenes.

In certain embodiments, the hydrocarbon feed supplied to the alkyl-demethylation zone can comprise C9+ aromatic hydrocarbons including ethylmethylbenzenes, C3-alkyl substituted benzenes, indane, trimethylbenzenes, C4-alkyl substituted benzenes, methylindanes, tetramethylbenzenes, tetralin, methyltetralins, and the like, at various concentrations. In certain embodiments, the concentration of C9+aromatic hydrocarbons bearing a C2+hydrocarbyl substitute in the feed provided to the alkyl-demethylation zone can range from cxl to cx2 wt %, based on the total weight of the C9+aromatic hydrocarbons contained in the feed, wherein cxl and cx2 can be, independently, e.g., 10, 15, 20, 25, 30, 35, 40, 45, 50, 55, 60, 65, 70, 75, 80, 85, or 90, as long as cx1<cx2. The processes of this disclosure can be particularly advantageous when used to process such streams comprising high concentrations of C9+ aromatic hydrocarbons bearing a C2+ substitute such as at ≥30 wt %, ≥40 wt %, ≥50 wt %, ≥60 wt %, ≥70 wt %, or ≥80 wt %, based on the weight of all C9+ aromatic hydrocarbons in the feed. In the present disclosure, C9+ aromatic hydrocarbons bearing a C2+ substitute can be conveniently converted into useful methylated aromatic hydrocarbons such as toluene, xylenes, and trimethylbenzenes. Through processes known to those having ordinary skill in the art, the toluene can be converted into additional quantities of xylenes, particularly p-xylene, via methylation with methanol and/or dimethyl ether, toluene disproportionation, and transalkylation with C9+ aromatic hydrocarbons, particularly methylated aromatic hydrocarbons such as trimethylbenzenes and tetramethylbenzenes. C9+ methylated aromatic hydrocarbons, including trimethylbenzenes, tetramethylbenzenes, and the like, can be converted into additional quantities of xylenes, particularly p-xylene, via transalkylation with benzene and/or toluene as well.

The alkyl-demethylation step is preferably carried out in the presence of molecular hydrogen co-fed into the alkyl-demethylation zone. The methyl group(s) removed in the alkyl-demethylation step in the alkyl-demethylation zone may be converted into light hydrocarbons such as methane in the presence of the molecular hydrogen. Hydrogen partial pressures in the alkyl-demethylation zone may range from 50 to 2,500 kilopascal, absolute, for example.

The processes of this disclosure can include a reaction taking place in one or more alkyl-demethylation zones. An alkyl-demethylation zone can include a portion of a reactor, a full reactor, or multiple reactors, which may be in series or parallel. Where multiple alkyl-demethylation zones are present in the processes of this disclosure, the catalysts and conditions for promoting alkyl-demethylation in them may be the same or different.

The alkyl-demethylation conditions (e.g., the first, second, third, fourth, fifth, sixth, and seventh or more alkyl-demethylation conditions) in the alkyl-demethylation zone(s) can vary widely, depending on the composition of the feed being subjected to alkyl-demethylation. Even in a single alkyl-demethylation zone, the alkyl-demethylation conditions can vary widely during a production campaign, or from one production campaign to another. Thus, the alkyl-demethylation conditions in the alkyl-demethylation zone(s) can include a temperature in a range from t1 to t2° C., wherein t1 and t2 can be, independently, e.g., 180, 200, 220, 240, 250, 260, 280, 300, 320, 340, 350, 360, 380, 400, 420, 440, 450, 460, 480, or 500, as long as t1<t2. The alkyl-demethylation conditions in the alkyl-demethylation zone(s) can include an absolute pressure in a range from p1 to p2 kilopascal, wherein p1 and p2 can be, independently, e.g., 100, 150, 200, 250, 300, 350, 400, 450, 500, 550, 600, 650, 700, 750, 800, 850, 900, 950, 1000, 1200, 1400, 1500, 1600, 1800, 2000, 2200, 2400, 2500, 3000, 3500, 4000, 4500, or 5000, as long as p1<p2. Partial pressure of hydrogen under the alkyl-demethylation conditions may range from h1 to h2 kilopascals, wherein h1 and h2 can be, independently, e.g., 50, 100, 150, 200, 250, 300, 350, 400, 450, 500, 600, 700, 800, 900, 1000, 1100, 1200, 1300, 1400, 1500, 1600, 1700, 1800, 1900, 2000, 2100, 2200, 2300, 2400, or 250, as long as h1<h2. Thus, depending on temperature and pressure conditions, the alkyl-demethylation conditions in the alkyl-demethylation zone(s) can be such that the aromatic hydrocarbons in the alkyl-demethylation zone(s) are substantially in vapor phase, substantially in liquid phase, or a mixed phase comprising liquid phase and vapor phase in any ratio. The alkyl-demethylation conditions can include a molecular hydrogen to C6+ aromatic hydrocarbons molar ratio in a range from r1 to r2, where r1 and r2 can be, independently, e.g., 0.1, 0.2, 0.3, 0.4, 0.5, 1, 2, 3, 4, 5, 6, 7, 8, 9, 10, 11, 12, 13, 14, 15, 16, 17, 18, 19, or 20, as long as r1<r2. The alkyl-demethylation conditions can further include a liquid weight hourly space velocity (“WHSV”) in a range from w1 to w2, where w1 and w2 can be, independently, e.g., 0.1, 0.2, 0.4, 0.5, 0.6, 0.8, 1, 2, 4, 5, 6, 8, 10, 12, 14, 15, 16, 18, or 20, as long as w1<w2.

Specifically, with respect to a C8 aromatic hydrocarbon stream comprising a majority by weight, or consisting essentially of, xylenes and ethylbenzene, preferably at least 5 wt % ethylbenzene based on a total weight of C6+ aromatic hydrocarbon-containing stream, such as a p-xylene depleted stream produced from a p-xylene separation process, the alkyl-demethylation conditions in the alkyl-demethylation zone(s) can include a temperature in a range from t3 to t4° C., wherein t3 and t4 can be, independently, e.g., 180, 200, 220, 240, 250, 260, 280, 300, 320, 340, 350, 360, 380, 400, 420, 440, 450, 460, 480, or 500, as long as t3<t4. The alkyl-demethylation conditions in the alkyl-demethylation zone(s) can include an absolute pressure in a range from p3 to p4 kilopascal, wherein p3 and p4 can be, independently, e.g., 100, 150, 200, 250, 300 350, 400, 450, 500, 550, 600, 650, 700, 750, 800, 850, 900, 950, 1000, 1200, 1400, 1500, 1600, 1800, 2000, 2200, 2400, 2500, 3000, 3500, 4000, 4500, or 5000, as long as p3<p4. Partial pressure of hydrogen under the alkyl-demethylation conditions may range from h1 to h2 kilopascals, wherein h1 and h2 can be, independently, e.g., 50, 100, 150, 200, 250, 300, 350, 400, 450, 500, 600, 700, 800, 900, 1000, 1100, 1200, 1300, 1400, 1500, 1600, 1700, 1800, 1900, 2000, 2100, 2200, 2300, 2400, or 250, as long as h1<h2. Thus, depending on temperature and pressure conditions, the alkyl-demethylation conditions in the alkyl-demethylation zone(s) can be such that the C8 aromatic hydrocarbons are substantially in vapor phase, substantially in liquid phase, or a mixed phase comprising liquid phase and vapor phase in any ratio. The alkyl-demethylation conditions can include a molecular hydrogen to C6+ aromatic hydrocarbons molar ratio in the alkyl-demethylation zone(s) in a range from r1 to r2, where r1 and r2 can be, independently, e.g., 0.1, 0.2, 0.3, 0.4, 0.5, 1, 2, 3, 4, 5, 6, 7, 8, 9, 10, 12, 14, 15, 16, 18, or 20, as long as r1<r2. The alkyl-demethylation conditions in the alkyl-demethylation zone(s) can further include a weight hourly space velocity (“WHSV”) in a range from w1 to w2, where w1 and w2 can be, independently, e.g., 0.1, 0.2, 0.4, 0.5, 0.6, 0.8, 1, 2, 4, 5, 6, 8, 10, 12, 14, 15, 16, 18, or 20, as long as w1<w2.

Specifically, with respect to a C9+ aromatic hydrocarbons stream comprising a majority by weight, or consisting essentially of, C9+ aromatic hydrocarbons, such as a C9+ aromatic hydrocarbons stream produced from p-xylene separation process or from another aromatic hydrocarbon source, preferably comprising at least 80 wt % C9+ aromatic hydrocarbons and at least 20 wt % C2+-hydrocarbyl-substituted C9+ aromatic hydrocarbons based on a total weight of C6+ aromatic hydrocarbon-containing stream, the alkyl-demethylation conditions in the alkyl-demethylation zone(s) can include a temperature in a range from t3 to t4° C., wherein t3 and t4 can be, independently, e.g., 180, 200, 220, 240, 250, 260, 280, 300, 320, 340, 350, 360, 380, 400, 420, 440, 450, 460, 480, or 500, as long as t3<t4. The alkyl-demethylation conditions in the alkyl-demethylation zone(s) can include an absolute pressure in a range from p3 to p4 kilopascal, wherein p3 and p4 can be, independently, e.g., 100, 150, 200, 250, 300 350, 400, 450, 500, 550, 600, 650, 700, 750, 800, 850, 900, 950, 1000, 1200, 1400, 1500, 1600, 1800, 2000, 2200, 2400, 2500, 3000, 3500, 4000, 4500, or 5000, as long as p3<p4. Partial pressure of hydrogen under the alkyl-demethylation conditions may range from h1 to h2 kilopascals, wherein h1 and h2 can be, independently, e.g., 50, 100, 150, 200, 250, 300, 350, 400, 450, 500, 600, 700, 800, 900, 1000, 1100, 1200, 1300, 1400, 1500, 1600, 1700, 1800, 1900, 2000, 2100, 2200, 2300, 2400, or 250, as long as h1<h2. Thus, depending on temperature and pressure conditions, the alkyl-demethylation conditions in the alkyl-demethylation zone(s) can be such that the C9+ aromatic hydrocarbons are substantially in vapor phase, substantially in liquid phase, or a mixed phase comprising liquid phase and vapor phase in any ratio. The alkyl-demethylation conditions can include a molecular hydrogen to C6+ aromatic hydrocarbons molar ratio in the alkyl-demethylation zone(s) in a range from r1 to r2, where r1 and r2 can be, independently, e.g., 0.1, 0.2, 0.3, 0.4, 0.5, 1, 2, 3, 4, 5, 6, 7, 8, 9, 10, 12, 14, 15, 16, 18, or 20, as long as r1<r2. The alkyl-demethylation conditions in the alkyl-demethylation zone(s) can further include a weight hourly space velocity (“WHSV”) in a range from w1 to w2, where w1 and w2 can be, independently, e.g., 0.1, 0.2, 0.4, 0.5, 0.6, 0.8, 1, 2, 4, 5, 6, 8, 10, 12, 14, 15, 16, 18, or 20, as long as w1<w2.

Accordingly, processes for converting C2+-hydrocarbyl-substituted aromatic hydrocarbons via alkyl-demethylation according to the present disclosure may comprise: providing a C6+ aromatic hydrocarbon-containing stream comprising the C2+-hydrocarbyl-substituted aromatic hydrocarbon, wherein the C2+-hydrocarbyl-substituted aromatic hydrocarbon has (i) a C2+ alkyl substitute attached to an aromatic ring therein and/or (ii) an aliphatic ring annelated to an aromatic ring therein; and contacting the C6+ aromatic hydrocarbon-containing stream comprising the C2+-hydrocarbyl-substituted aromatic hydrocarbon with a catalyst composition of the present disclosure in an alkyl-demethylation zone under alkyl-demethylation conditions effective to convert at least a portion of the C2+-hydrocarbyl-substituted aromatic hydrocarbon to an alkyl-demethylated aromatic hydrocarbon comprising at least one methyl substitute to obtain a first alkyl-demethylated effluent exiting the alkyl-demethylation zone. As discussed further hereinbelow, the catalyst composition may comprise an oxide support material having a surface acidity below a threshold level, preferably a surface acidity no greater than that of gamma alumina, and a transition metal element dispersed upon the oxide support material, wherein the catalyst composition exhibits a hydrogen chemisorption value of at least 15% as a measure of the transition metal element dispersion upon the oxide support material.

At least a portion of the transition metal element may be in elemental form when disposed upon the support material and the hydrogen chemisorption is measured. The transition metal element may be substantially in elemental form, or a mixture of the transition metal element in element form and a non-elemental form (e.g., a transition metal oxide or transition metal salt) may be present upon the support material.

When measuring hydrogen chemisorption, the following procedure may be used: Roughly 250 mg of a catalyst composition precursor may be loaded into an Autochem 2920 instrument, which may then be ramped to 250° C. at 10° C./min under Ar flow, followed by a 30 minute hold at this temperature to drive off any moisture and other volatile species. After this drying step, 10% H₂ may be introduced to the system, and the catalyst composition precursor is heated to 450° C. at 10° C./min, followed by a 60 minute hold to reduce the transition metal and form an active catalyst composition. The catalyst composition is then cooled to 40° C. and 0.5 mL of H2 is pulsed over the catalyst to allow H₂ to absorb onto the surface of the transition metal. The pulses are repeated until no further H₂ adsorption is detected via TCD. The dispersion is then calculated using the amount of H₂ adsorbed on the metal and the loading of metal in the catalyst composition.

Suitable oxide support materials for forming the catalyst compositions of the present disclosure include alkaline earth metal oxides, silica, a composite of an alkaline earth metal oxide and Al₂O₃, a composite of ZnO and Al₂O₃, a lanthanide oxide, a composite of a lanthanide oxide and Al₂O₃, and combinations or mixtures of two or more thereof. More specific examples of suitable oxide support materials may include, for example, CaO, MgO, SrO, silica (preferably precipitated silica), a composite of MgO and Al₂O₃, such as a spinel formed from calcined hydrotalcite, and combinations and mixtures of two or more thereof. Suitable oxide support materials may further or alternately exhibit an alpha value of no greater than 2.5, preferably no greater than 2.0, and even more preferably no greater than 1.5. Measurement of the alpha value was performed based upon the conversion of n-hexane into lighter hydrocarbons. A reactor was loaded with about 1 cc of catalyst, and n-hexane was sparged into a stream of helium passed through the reactor at elevated temperature (1000° F.). An in-line GC was used to analyze the reactor effluent to determine the amount of hexane converted to products. The conversion of n-hexane by the catalyst relative to that produced by alumina under similar conditions provides the alpha value.

Contacting to obtain an alkyl-demethylated aromatic hydrocarbon according to the present disclosure may exhibit at least one of the following: a positive methyl gain, a C2+-alkyl group conversion in a range from 30% to 100%, and an aromatic ring loss of no greater than 3%. More specific examples of the contacting steps may include those that exhibit at least one of the following: a xylene loss in a range from 0% to 15%, an ethylbenzene conversion in a range from 30% to 100%, a positive methyl gain, and an aromatic ring loss no greater than 3%. Other more specific examples of the contacting steps may include those that exhibit at least one of the following: a trimethylbenzenes loss in a range from 0% to 20%, a C2+-alkyl-substituted C9+ aromatic hydrocarbons conversion in a range from 30% to 100%, a positive methyl gain, and an aromatic ring loss no greater than 3%, more particularly instances in which contacting further exhibits at least one of the following: an ethyl group conversion from 30% to 100%, and a C3-alkyl group conversion from 30% to 100%.

Methyl gain may be determined by subtracting the amount of methyl groups in the product from the amount of methyl groups in the C6+-aromatic hydrocarbon-containing stream divided by the mount of methyl groups in the C6+-aromatic hydrocarbon-containing stream. A positive value represents a gain in methyl groups during alkyl-demethylation, and a negative value is reflective of a loss of methyl groups during alkyl-demethylation. Ring loss may be determined by subtracting the amount of aromatic rings in the product from the amount of aromatic rings in the C6+-aromatic hydrocarbon-containing stream and dividing the difference by the number of aromatic rings in the C6+-aromatic hydrocarbon-containing stream. Xylene loss, ethylbenzene conversion, trimethylbenzenes conversion, and C2+-alkyl-substituted C9+ aromatic hydrocarbon conversion are determined similarly by dividing the difference in value for product and stream by the value in the C6+-aromatic hydrocarbon-containing stream.

Suitable alkyl-demethylation conditions may comprise at least one of the following: a presence of molecular hydrogen in the alkyl-demethylation zone at a partial pressure of hydrogen in a range from 50 to 2,500 kilopascal absolute, a temperature in a range from 180 to 500° C., an absolute total pressure in a range from 100 to 5,000 kilopascal, a WHSV in a range from 0.1 to 20 hour⁻¹, and a molar ratio of molecular hydrogen to the C6+ aromatic hydrocarbon-containing stream in a range from 0.1 to 10.

In any embodiment, the C6+ aromatic hydrocarbon-containing stream may comprise at least 80 wt % C8 aromatic hydrocarbons, and at least 5 wt % ethylbenzene based on a total weight of the C6+ aromatic hydrocarbon-containing stream. In any embodiment, the C6+ aromatic hydrocarbon-containing stream may comprise at least 80 wt % C9+ aromatic hydrocarbons, and at least 20 wt % C2+-hydrocarbyl-substituted C9+ aromatic hydrocarbons, based on a total weight of the C6+ aromatic hydrocarbon-containing stream. The C2+-hydrocarbyl-substituted aromatic hydrocarbons in either of these C6+ aromatic hydrocarbon-containing streams may be present substantially in vapor phase in the alkyl-demethylation zone, as defined herein.

### Catalyst Compositions Suitable for Performing Alkyl-Demethylation

The catalyst composition may comprise an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and combinations and mixtures of two or more thereof, a transition metal element dispersed upon the oxide support material, wherein the catalyst composition exhibits a hydrogen chemisorption value of at least 15%. Suitable oxide support materials may include those exhibiting an alpha value of no greater than 2.5, preferably no greater than 2.0, or more preferably no greater than 1.5.

Without being bound by any theory or mechanism, the catalyst compositions of the present disclosure may feature the transition metal element well dispersed upon the oxide support material, wherein the extent of metal dispersion may be correlated through the hydrogen chemisorption value. Higher hydrogen chemisorption values are believed to be reflective of increased metal dispersion. Lower surface acidity values are believed to lead to increased metal dispersion according to the disclosure herein. Preferably, the oxide support material used in the catalyst compositions of the present disclosure are substantially free of zeolites and alumina, since these materials have high surface acidity values. Oxide composites in which Al₂O₃ is not present as a discrete phase, such as a spinel formed from calcined hydrotalcite and similar oxide composites, may be suitable for use in the catalyst compositions of the present disclosure, and such composites may be present even when zeolites and alumina are otherwise excluded from the oxide support material. More particular examples of oxide support materials suitable for use in the disclosure herein include those comprising a member selected from the group consisting of CaO, MgO, SrO, silica (preferably precipitated silica), a composite of MgO and Al₂O₃, and mixtures and combinations of two or more thereof.

The amount of the oxide support materials in the catalyst compositions disclosed herein can range from c(s)1 to c(s)2 wt %, where c(s)1 and c(s2)2 can be, independently, e.g., 2, 4, 5, 6, 8, 10, 15, 20, 25, 30, 35, 40, 45, 50, 55, 60, 65, 70, 75, 80, 85, 90, 95, 96, 97, 98, or 99, as long as c(s)1<c(s)2.

Preferably, the oxide support material in the catalyst compositions disclosed herein may exhibit a BET surface area of at least 25 m²/g, including 25 m²/g to 500 m²/g, or 25 m²/g to 200 m²/g, or 25 m²/g to 100 m²/g, or 50 m²/g to 300 m²/g, or 100 m²/g to 400 m²/g. To perform the BT surface area measurements, a Micromeritcs TriStar 3000 instrument may be used. The sample may be heated at 350° C. and degassed using a vacuum pump, and then placed in He flow for several hours. The sample is then cooled to liquid nitrogen temperature, and nitrogen is physically adsorbed onto the sample. Desorption of the nitrogen may afford a Langmuir curve, from which the surface area can be derived.

Preferably, the catalyst composition may exhibit a hydrogen chemisorption value of at least 50%, more preferably a hydrogen chemisorption value of at least 90%, and still more preferably a hydrogen chemisorption value of 100% based on a theoretical limit.

Catalyst compositions of the present disclosure may feature at least a portion of the transition metal element in an elemental state, which may be mixed with a non-elemental state (e.g., a transition metal oxide or salt) or the transition metal element may be present in a substantially elemental state. When present in a substantially elemental state, the transition metal element is not bound to other metal elements or to ligand atoms, and/or with the transition metal element is not admixed with appreciable amounts of other forms of the transition metal element. The substantially elemental state may be produced in situ under alkyl-demethylation conditions, as specified herein. Additionally or alternately, the elemental state may be produced upon exposing the transition metal element to hydrogen gas at a suitable temperature, either as a separate catalyst activation step and/or a catalyst activation step taking place in situ in an alkyl-demethylation zone. Thus, when the transition metal element is not dispersed in an elemental state upon the oxide support material (i.e., within a catalyst precursor composition), suitable reducing conditions may be utilized to convert the non-elemental transition metal element into an elemental state for promoting an alkyl-demethylation reaction according to the disclosure herein.

The transition metal element present in the catalyst compositions (or catalyst composition precursors) of the present disclosure may comprise one or more transition metal elements selected from Groups 8-10 of the Periodic Table, specifically Fe, Co, Ni, Ru, Rh, Pd, Re, Os, Ir, Pt, and mixtures and combinations of two or more thereof. Preferably, the transition metal element may comprise one or more transition metal elements selected from the group consisting of Rh, Pd, Ir, Pt, and mixtures and combinations of two or more thereof. In more particular examples of the present disclosure, the transition metal element may comprise or consist essentially of Rh.

The concentration of the transition metal element, based on the total weight of the catalyst composition, can range from c(m1)1 to c(m1)2 wt %, where c(m1)1 and c(m1)2 can be, independently, e.g., 0.01, 0.02, 0.04, 0.05, 0.06, 0.08, 0.1, 0.2, 0.4, 0.5, 0.6, 0.8, 1, 2, 4, 5, 6, 8, 10, as long as c(m1)1<c(m1)2. Particular examples may include those in which the catalyst composition comprises 0.01 to 5 wt % transition metal element, preferably Rh, expressed as a weight percentage of the transition metal element in an elemental state relative to a total weight of the catalyst composition. More specific examples of the catalyst compositions may include those in which the catalyst composition comprises 0.1 to 2 wt % transition metal element, preferably Rh, expressed as a weight percentage of the transition metal element in an elemental state relative to a total weight of the catalyst composition.

The catalyst compositions may further comprise an optional promoter selected from Groups 1 and 2 metal elements, and combination and mixtures thereof. The amount of the promoter can range from c(p)1 to c(p)2 wt %, based on the total weight of the catalyst compositions, where c(p)1 and c(p)2 can be, independently, e.g., 0.01, 0.02, 0.04, 0.05, 0.06, 0.08, 0.1, 0.2, 0.4, 0.5, 0.6, 0.8, 1, 2, 4, 5, 6, 8, 10, as long as c(p)1<c(p)2. Preferably, the optional promoter may comprise a metal selected from Li, Na, K, Cs, Mg, Ca, Ba, and mixtures and combinations of two or more thereof. The promoters, when used, may enhance the performance of the catalyst compositions, particularly in terms of activity and selectivity for alkyl-demethylation.

### Processes for Making the Catalyst Compositions and Catalyst Composition Precursors

Processes for making a catalyst composition suitable for promoting alkyl-demethylation or a catalyst composition precursor thereof may comprise: providing an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and combinations and mixtures of two or more thereof; providing a source material of a transition metal element; dispersing the source material of the transition metal element on the oxide support material to obtain a catalyst composition precursor; and contacting the catalyst composition precursor with a reducing atmosphere, such as an atmosphere comprising hydrogen, under activating conditions to obtain the catalyst composition. Prior to being contacted with the reducing atmosphere, the transition metal element may be present in any form including, for example, an elemental form of the transition metal element, an oxide form of the transition metal element, a complex of the transition metal element, or a mixture or combination of any two or more or the foregoing.

Activating conditions comprising a reducing atmosphere suitable for promoting catalyst activation may include those featuring at least one of the following: a temperature in a range from 200° C. to 600° C.; a hydrogen partial pressure in a range from 5 to 200 kilopascal; and a total pressure in a range from 100 to 1,000 kilopascal.

Following activation under the reducing atmosphere to produce an elemental transition metal element, optionally admixed with another transition metal element form, the catalyst compositions may exhibit a hydrogen chemisorption value of at least 15%. Preferably, the catalyst compositions may exhibit a hydrogen chemisorption value of at least 50%, more preferably a hydrogen chemisorption value of at least 90% and still more preferably a hydrogen chemisorption value of 100% based on a theoretical limit.

Oxide composites, such as a spinel formed from calcined hydrotalcite, possess innate functionality capable of bonding a transition metal element without undergoing further surface modification. The bonding between the transition metal element and the oxide support material may promote dispersion of the transition metal element thereon. Other oxide support materials having low surface acidity values may lack innate capabilities for bonding a transition metal element. Such oxide support materials may be manipulated to promote bonding between the transition metal element and the oxide support material. Bonding may occur by virtue of at least partial deprotonation of the oxide support material, optionally accompanied by formation of surface hydroxyl groups, and/or by forming a bridging group comprising one or more bridging atoms between the transition metal element and the oxide support material.

Processes for forming a catalyst composition or a catalyst composition precursor may include those in which the oxide support material is provided in an at least partially deprotonated state. The oxide support material in the at least partially deprotonated state may be formed from an oxide support precursor, either in situ when contacting the transition metal element or prior to contacting the transition metal element. As such, providing the oxide support material according to such configurations of the present disclosure may comprise providing an oxide support precursor, and contacting the oxide support precursor with an alkaline solution to obtain the oxide support material, wherein the oxide support material or the oxide support precursor comprises surface hydroxyl groups. An oxide support precursor lacking surface hydroxyl groups may still form surface hydroxyl groups through equilibrium once the oxide support material is contacted with an aqueous solution, wherein deprotonation of the surface hydroxyl groups may occur if the aqueous solution is alkaline. After being contacted with the alkaline solution, at least a portion of the surface hydroxyl groups may be deprotonated to afford an oxide support material in a form capable of binding the transition metal element in a dispersed state, with silica being a representative example of an oxide support material that may be activated for transition metal element binding in such a manner The alkaline solution may comprise an alkaline aqueous solution in particular examples of the present disclosure and include ammonium hydroxide (NH₄OH), sodium hydroxide (NaOH), potassium hydroxide (KOH), or a mixture of two or more thereof. When used, excess NaOH or KOH may be washed from the surface of the oxide support material after forming an at least partially deprotonated surface. Preferably, an alkaline aqueous solution promoting conversion of the oxide support precursor into a form suitable for bonding a transition metal element may be a saturated ammonium hydroxide solution (concentrated aqueous ammonia).

Silica, such as precipitated silica, may represent a suitable oxide support precursor that may be effectively contacted with an alkaline solution to promote at least partial deprotonation for encouraging bonding of the transition metal element. Without full or partial surface deprotonation occurring, silica may otherwise have little affinity for bonding and dispersing a transition metal element effectively.

Once a suitable oxide support material has been provided, dispersing a source material upon the oxide support material may comprise: forming a liquid dispersion comprising the source material of the transition metal element and a liquid dispersant; contacting the oxide support material with the liquid dispersion of the source material of the transition metal element to obtain a mixture of the oxide support material and the source material; and drying the mixture of the oxide support material and the source material to form a dried mixture of the oxide support material and the source material. The liquid dispersion and the liquid dispersant thereof may comprise an aqueous liquid in particular process configurations, with water being a preferred aqueous liquid. The source material may comprise a salt of the transition metal element that is at least partially soluble in the liquid dispersion. In the case of an aqueous liquid dispersion, a metal salt that is at least partially soluble in water may be desirable. Aqueous solubility properties of metal salts of a transition metal element will be familiar to one having ordinary skill in the art, such that one having ordinary skill in the art will be able to choose a metal salt of a given transition metal having suitable solubility properties. In particular examples, suitable metal salts for practicing the disclosure herein may be a transition metal nitrate, a transition metal halide, or an organic salt of the transition metal element, many of which exhibit high aqueous solubility. Organic ligand complexes of the transition metal element may be used similarly as well.

Contacting the oxide support material with the liquid dispersion may comprise impregnating the oxide support material with the liquid dispersion via an incipient wetness technique. Incipient wetness impregnation involves adding just enough liquid solution of a metal salt to an oxide support material to fill the pores of the support completely without excess liquid being present. For high surface area silica supports, incipient wetting impregnation may be accomplished by adding —1.1 mL of solution for every gram of support.

Once the transition metal element has become impregnated within the oxide support material, the catalyst composition or a precursor thereof may be further calcined in air in certain process configurations to promote dispersion of the transition metal element. Specifically, in such process configurations, dispersing the source material of the transition metal element may comprise calcining the dried mixture of the oxide support material and the source material at a temperature in a range from 100° C. to 600° C. Calcining in this manner in air or a similar oxygen-containing atmosphere may convert the source material into an oxide form of the transition metal element.

Alternative process configurations for dispersing the transition metal element may include those in which the dried mixture of the oxide support material and the source material of the transition metal element are not calcined, preferably by heating at a temperature below 200° C., or more preferably by heating at a temperature below 100° C. Heating at such temperatures may take place in air or under an inert atmosphere, such as nitrogen. Such “non-calcined” processing of the catalyst composition may likewise promote dispersion of the transition metal element. Unlike calcination conditions, non-calcined processing of the catalyst composition or catalyst composition precursor may leave the source material at least partially in its original form (i.e., as a transition metal salt or transition metal complex) without being converted to an oxide form of the transition metal element.

In still another example, the transition metal element may be covalently linked to the oxide support material, such as silica, by a linking group provided by a linking agent. The linking agent may be bifunctional and provide heteroatoms for reacting with the oxide support material and the transition metal element. Examples of suitable linking agents may include, but are not limited to, an amino alcohol, an amino acid, a glycol, or a mixture of combination of two or more thereof. The linking agent may be present in the liquid dispersion of the source material and the liquid dispersant that is contacted with the oxide support material, thereby forming the linking group in situ when contacting the oxide support material. Alternately, the transition metal element may be reacted with a first functional group of the linking agent separately from the liquid dispersion, such that the second functional group of the linking agent remains free. Upon forming the liquid dispersion, the second functional group may then become bound to the oxide support material.

To facilitate a better understanding of the present disclosure, the following examples of preferred or representative embodiments are given. In no way should the following examples be read to limit, or to define, the scope of the invention.

### EXAMPLES

Catalytic reactions in the present disclosure were carried out in packed bed reactors using 0.5-10 g of catalyst precursor, sized through a 40-60 mesh sieve, and diluted with quartz or silicon carbide in a 1:1 or 1:2 ratio of catalyst precursor to diluent. Preparation of the catalyst precursors and catalytic reaction data are provided in the numbered examples below. The catalyst precursor was reduced prior to introducing an aromatic feed thereto. Reduction was performed by flowing hydrogen gas over the catalyst precursor for 3 hours at 450° C. Following reduction, the reactor was cooled to the specified run temperature and pressure before introducing the aromatic feed. The aromatic feed included a specified C2+ aromatic hydrocarbon combined with hydrogen, as further provided in the numbered examples below. Typical run conditions included a temperature of 320° C.-420° C., a pressure of 50-200 psig, a hydrogen:hydrocarbon molar ratio of 1:1 to 4:1, and a space velocity from 0.5-6 h⁻¹.

Two different aromatics feeds were processed in the numbered examples below. The first feed (Feed #1) was a typical xylenes isomerization feed comprising 87.5% xylenes and 12.5% ethylbenzene. The second feed (Feed #2) was a typical heavy aromatics (HAR) feed comprising 53% trimethylbenzenes, 21% methylethylbenzenes, and 26% other C8+ alkylaromatics.

Methyl gain is one parameter that was used to characterize the reaction products formed from the aromatic feeds after being catalytically processed. Methyl gain, which can be a positive or negative value, represents the increase or decrease in methyl group content of the reaction product in comparison to the feed. Methyl gain may be calculated with the expression (n1-n2)/n2*100%, wherein n1 is the number of moles of methyl group attached to an aromatic ring in the reaction product and n2 is the number of moles of methyl group attached to an aromatic ring in the feed provided to an alkyl-demethylation zone of the reactor. In the description of the examples below, Et conversion means the conversion of ethyl groups, Pr conversion means the conversion of propyl groups (n-propyl and i-propyl groups), EB conversion means ethylbenzene conversion, and TMB means trimethylbenzenes.

### Part A: Fabrication and Characterization of Catalyst Compositions

Example A1: Synthesis of Rh on Calcined Hydrotalcite. Rh was deposited on calcined hydrotalcite (calcined MG30, available from Sasol) by an incipient wetness method. Calcination of the hydrotalcite prior to Rh deposition resulted in formation of a spinel. The spinel formed following calcination of MG30 hydrotalcite (41.46% Al, 13.0% Mg) had a BET surface area of 196 m²/g, a 0.45 cm³/g pore volume, and a pore size centered at 66 Å. As an example preparation, 1% Rh was loaded as follows: 2.007 g of rhodium nitrate solution containing 10.066 wt % Rh was diluted with distilled water. The total volume of the rhodium nitrate solution after dilution was 8.55 ml, a volume which represented 95% of the solution absorption capacity of 20 g of a calcined MG30 hydrotalcite extrudate. After Rh impregnation by incipient wetting, the sample was dried in air at 250° F. (120° C.) for 16 hrs, and then further calcined in air at 572° F. (300° C.) for 1 hr. The furnace was ramped at rate of 5° F./min. During calcination, the air flow was adjusted at 5 volume/catalyst volume/minute. Catalyst precursors having Rh loadings of 0.06, 0.13, 0.25, 0.50, 0.75, and 1.25 wt % were prepared in a similar manner.

Catalyst precursor samples were also made from a spinel formed through calcination of MG70 hydrotalcite (Sasol, 21.97% A1, 30.53% Mg). The spinel formed through calcination of MG70 hydrotalcite had a BET surface area of 197 m²/g, a 0.30 cm³/g pore volume, and a pore size centered at 56 Å. In general, this oxide support afforded similar catalytic performance to that provided by the spinel formed through calcination of MG30 hydrotalcite (data not shown below).

Example A2: Synthesis of Rh on Alumina (Comparative). Comparative samples comprising 0.5% or 1% Rh on Al₂O₃ were also prepared in a similar manner to that described above.

Characterization. Metal dispersion within the catalyst precursors of Examples A1 and A2 was determined through H₂ chemisorption at 40° C., data for which is shown in Table 1 below. The catalyst precursors were first activated under H₂ at high temperature, followed by cooling and then measuring hydrogen uptake upon the reduced metal.

TABLE 1

| | | | | Metal |
|---|---|---|---|---|
| | | Catalyst Composition | | Dispersion |
| No. | Example No. | Rh (%) | Support | (%) |
| 1 | A1 | 1.25 | Calcined MG30 | 32.7 |
| 2 | A1 | 1.00 | Calcined MG30 | 33.5 |
| 3 | A1 | 0.75 | Calcined MG30 | 25.9 |
| 4 | A1 | 0.50 | Calcined MG30 | not measured |
| 5 | A1 | 0.25 | Calcined MG30 | 98.6 |
| 6 | A2 (Comparative) | 0.50 | Alumina | 41.0 |
| 7 | A2 (Comparative) | 1.00 | Alumina | 46.8 |

As shown, the metal dispersion on the spinel formed through hydrotalcite calcination was only slightly lower than that obtained on alumina, and in one case, the metal dispersion was much higher. In spite of the lower metal dispersion, the catalyst performance afforded by the calcined hydrotalcite support was considerably better, as shown the catalyst performance experiments below.

Example A3: Synthesis of Rh on Silica Using an Amine Linker. A solution of rhodium nitrate was mixed with a solution of triethanolamine or arginine linker in a 1:20 molar ratio (mol. Rh:mol. linker) in water. The combined solution was equilibrated for 24 hours and then used to impregnate a silica support with just enough liquid to completely fill the void volume of the support by incipient wetness. The sample was then dried in a vacuum oven at 70° C. overnight and calcined in air at 300° C. for 1 hour. Catalyst precursor samples having 0.5% or 1.0% Rh loading by weight were synthesized using an arginine linker in a similar manner.

Example A4: Synthesis of Rh on Silica Using Ammonia Treatment. Rhodium nitrate (500 mg) was dissolved in a 30% wt/vol solution (30 mL) of ammonia, and the combined solution was equilibrated for 48 hours. A portion of the combined solution was then used to impregnate a silica support with just enough volume to completely fill the pore volume of the support by incipient wetness. The sample was then dried in a vacuum oven at 70° C. overnight and calcined in air at 300° C. for 1 hour. Catalyst precursor samples having 0.5% or 1.0% Rh loading by weight were synthesized using the foregoing method.

Example A5: Synthesis of Rh on Silica Without High-Temperature Calcination. Rhodium nitrate (500 mg) was dissolved in water (30 mL), and the solution was then used to impregnate a silica support with just enough volume to completely fill the pore volume of the support by incipient wetness. The sample was then dried in a vacuum oven at 70° C. overnight. Catalyst precursor samples having 0.5% or 1.0% Rh loading by weight were synthesized using the foregoing method.

Example A6: Synthesis of Rh on Silica (Comparative). Rhodium nitrate (500 mg) was dissolved in water (30 mL), and the solution was impregnated into a silica support with just enough volume to completely fill the pore volume of the support by incipient wetness. The sample was then dried at 70° C. overnight in a vacuum oven and calcined in air at 300° C. for 1 hour. A catalyst precursor sample having 1.0% Rh loading by weight was synthesized using the foregoing method.

Characterization. Metal dispersion within the catalyst precursors of Examples A3-A6 was determined through H2 chemisorption at 40° C., which is shown in Table 2 below. The catalyst precursors were first activated under H₂ at high temperature, followed by cooling and then measuring hydrogen uptake upon the reduced metal.

TABLE 2

| | | | Metal |
|---|---|---|---|
| | | | Dispersion |
| No. | Example No. | Catalyst Precursor | (%) |
| 1 | A3 | Rh on silica, triethanolamine | 16 |
| 2 | A3 | Rh on silica, arginine | 45 |
| 3 | A4 | Rh on silica, ammonia | 77 |
| 4 | A5 | Rh on silica, no calcination | 55 |
| 5 | A6 (Comparative) | Rh on silica, calcined | 9 |

As shown in Table 2, all of the catalyst precursor samples prepared with a silica support according to the present disclosure exhibited a significantly increased extent of metal dispersion, as compared to the calcined Comparative Example (Example A6). In contrast to the catalyst precursors of Examples A3 to A5, the comparative catalyst precursor of Example A6 was nearly inactive for performing aromatic demethylation, even at 350° C. or 400° C., as shown in the data below. The low activity of the comparative catalyst precursor of Example A6 is believed to result from its low metal dispersion.

### Part B: Evaluation of Catalyst Compositions for Alkyl-Demethylation

Example B1: Catalyst Performance of Rh on Calcined Hydrotalcite for Xylenes/Ethylbenzene Conversion. Feed #1 was treated with the catalyst precursor of Table 1, Entry 4 (0.50% Rh on calcined hydrotalcite) under the general conditions specified above for catalyst activation and reaction. As a comparative example, the catalyst precursor of Table 1, Entry 6 was used for conversion of Feed #1 under the general conditions specified above for catalyst activation and reaction.

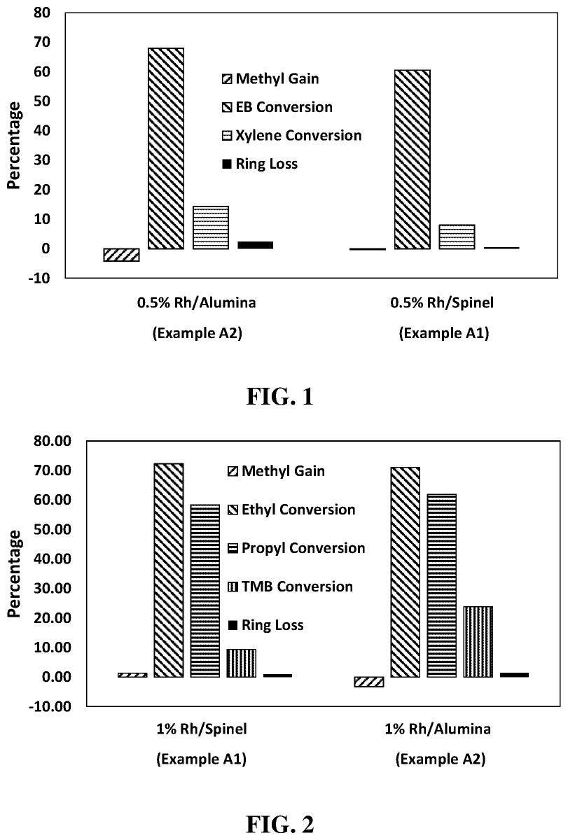

FIG. 1 is a graph showing illustrative conversion performance for 0.5% Rh on a spinel formed from calcined hydrotalcite in comparison to 0.5% Rh on alumina for catalytic treatment of xylenes/ethylbenzene. The reaction temperature for 0.5% Rh on alumina was 360° C., the pressure was 100 psig, the WHSV was 5 hr⁻¹, and the hydrogen:feed molar ratio was 2:1. For 0.5% Rh on calcined hydrotalcite, the reaction temperature was 380° C. and the other conditions were the same. As shown in Table 3, both catalyst precursors afforded a high extent of conversion of ethylbenzene (61% versus 68% for the calcined hydrotalcite sample and the comparative catalyst precursor, respectively). However, the comparative catalyst precursor led to much more significant xylene conversion (14% versus 8%) and aromatic ring loss (2.5% versus 0.5%), which is believed to result from the surface acidity of the alumina. In addition, the comparative catalyst precursor led to a significant loss of methyl groups (methyl gain=−4.2%), whereas the Rh on calcined hydrotalcite catalyst precursor provided a much less significant methyl group loss (methyl gain =−0.3%). The improved methyl gain and ring loss performance of the 0.5% Rh on calcined hydrotalcite catalyst precursor was realized in spite of a higher reaction temperature being used for this catalyst precursor sample.

Additional performance data for 0.5% Rh on the spinel formed from calcined hydrotalcite in comparison to 0.5% Rh on alumina for xylenes/ethylbenzene conversion under different reaction conditions is specified in Table 3 below. The pressure was 100 psig and the H₂:feed molar ratio was 2:1 (mol/mol) in all cases. As shown, the 0.5% Rh on spinel (e.g., calcined hydrotalcite) catalyst composition led to catalytic performance superior to that of 0.5% Rh on alumina in nearly every aspect under the tested reaction conditions. In particular, the 0.5% Rh on calcined hydrotalcite catalyst composition afforded less conversion of xylenes and less aromatic ring loss, which is reflected in the superior methyl gain value for this catalyst composition.

Example B2: Catalyst Performance of Rh on Calcined Hydrotalcite for HAR Conversion. Feed #2 was treated with the catalyst composition of Table 1, Entry 2 (1.00% Rh on calcined hydrotalcite) under the general conditions specified above for catalyst activation and reaction. As a comparative example, the catalyst composition of Table 1, Entry 7 was used for conversion of Feed #2 under the general conditions specified above for catalyst activation and reaction.

FIG. 2 is a graph showing illustrative conversion performance for 1.0% Rh on a spinel formed from calcined hydrotalcite in comparison to 1.0% Rh on alumina for catalytic treatment of a HAR feed. The reaction temperature for 1.0% Rh on alumina was 370° C., the pressure was 100 psig, the WHSV was 4 hr⁻¹, and the hydrogen:feed molar ratio was 2:1. For 1.0% Rh on calcined hydrotalcite, the WHSV was 6 hr⁻ and the other conditions were the same. As shown, both catalyst compositions afforded a high extent of conversion of ethyl and propyl groups, but the 1.0% Rh on alumina catalyst composition led to more extensive trimethylbenzenes conversion and higher aromatic ring loss than did the 1.0% Rh on calcined hydrotalcite catalyst composition. Under the testing conditions, the 1.0% Rh on calcined hydrotalcite catalyst composition afforded a positive methyl gain, in contrast to the performance of the 1.0% Rh on alumina catalyst composition, in spite of the higher reaction temperature used for this catalyst composition sample.

Additional performance data for 1.0% Rh on calcined hydrotalcite in comparison to 1.0% Rh on alumina for HAR conversion under different reaction conditions is specified in Table 4 below. The pressure was 100 psig and the H₂:feed molar ratio was 2:1 (mol/mol) in all cases. Under all of the tested conditions, the Rh on calcined hydrotalcite catalyst composition outperformed the corresponding Rh on alumina catalyst composition. On account of the low trimethylbenzenes conversion and low ring loss values, the Rh on calcined hydrotalcite catalyst compositions led to high methyl gain values.

Example B3: Catalyst Performance of Rh on Silica for Xylenes/Ethylbenzene Conversion. Feed #1 was treated with the silica-based catalyst compositions of Examples A3, A4 and A6 under the general conditions specified above for catalyst activation and reaction. As an additional comparative example, a 1% Rh on alumina catalyst composition (Example A2) was also tested under similar activation and reaction conditions. Performance data for the silica-based catalyst composition xylenes/ethylbenzene conversion under different reaction conditions is specified in Table 5 below. The pressure was 100 psig and the H₂:feed molar ratio was 2:1 (mol/mol) in all cases. As shown in Table 5, the silica-based catalyst compositions having increased metal dispersion exhibited superior performance relative to the comparative samples. The less negative methyl gain for the catalyst compositions of Examples A3 and A4, in comparison to the alumina-based catalyst composition of Example A6, largely results from their high selectivity for ethylbenzene conversion and their tendency to produce a low ring loss and little xylenes conversion.

Example B4: Catalyst Performance of Rh on Silica for HAR Conversion. Feed #2 was treated with the silica-based catalyst composition of Example A5 under the general conditions specified above for catalyst activation and reaction. As a comparative example, a 1% Rh on alumina catalyst composition (Example A2) was also tested under similar activation and reaction conditions. Performance data for the silica-based catalyst composition xylenes/ethylbenzene conversion under different reaction conditions is specified in Table 6 below. The pressure was 100 psig and the H₂:feed molar ratio was 2:1 (mol/mol) in all cases. As shown in Table 6, the silica-based catalyst compositions afforded positive methyl gains under several different reaction conditions, whereas the alumina-based catalyst composition did not. The silica-based catalyst compositions afforded similar ethyl and propyl conversion rates, while leading to less trimethylbenzenes and aromatic ring loss under similar reaction conditions, thereby producing a net methyl gain.

TABLE 3

| Catalyst Composition | | | Temperature | WHSV | Me Gain | EB Conversion | Xylene Conversion | Ring Loss |
|---|---|---|---|---|---|---|---|---|
| Example No. | Rh (%) | Support | (° C.) | (hr^{-l}) | (%) | (%) | (%) | (%) |
| A2 (Comparative) | 0.5 | Alumina | 350 | 5 | −4.64 | 66.09 | 13.08 | 3.01 |
| A2 (Comparative) | 0.5 | Alumina | 360 | 5 | −4.17 | 67.96 | 14.34 | 2.46 |
| A2 (Comparative) | 0.5 | Alumina | 340 | 2.5 | −5.82 | 63.36 | 14.46 | 6.77 |
| A2 (Comparative) | 0.5 | Alumina | 350 | 2.5 | −5.7 | 72.84 | 16.65 | 4.47 |
| A1 | 0.5 | Spinel | 350 | 5 | −1.98 | 37.2 | 5.96 | 2.69 |
| A1 | 0.5 | Spinel | 360 | 5 | −0.9 | 41.27 | 5.64 | 1.54 |
| A1 | 0.5 | Spinel | 370 | 5 | −0.26 | 49.31 | 6.18 | 0.82 |
| A1 | 0.5 | Spinel | 380 | 5 | −0.31 | 60.53 | 8 | 0.49 |

TABLE 4

| Catalyst Composition | | | Temperature | WHSV | Me Gain | Et Conversion | Pr Conversion | TMB Conversion | Ring Loss |
|---|---|---|---|---|---|---|---|---|---|
| Example No. | Rh (%) | Support | (° C.) | (hr^{-l}) | (%) | (%) | (%) | (%) | (%) |
| A2 (Comparative) | 1.0 | Alumina | 340 | 4 | −3.62 | 39.81 | 35.87 | 12.35 | 4.35 |
| A2 (Comparative) | 1.0 | Alumina | 360 | 4 | −2.11 | 59.35 | 49.83 | 18.06 | 2.00 |
| A2 (Comparative) | 1.0 | Alumina | 370 | 4 | −3.23 | 71.07 | 61.92 | 23.88 | 1.48 |
| A2 (Comparative) | 1.0 | Alumina | 370 | 6 | −0.59 | 54.49 | 45.06 | 13.85 | 1.06 |
| A1 | 1.0 | Spinel | 330 | 4 | −0.18 | 30.28 | 23.74 | 2.26 | 2.14 |
| A1 | 1.0 | Spinel | 350 | 4 | 1.74 | 59.04 | 39.89 | 6.36 | 1.40 |
| A1 | 1.0 | Spinel | 360 | 4 | 1.56 | 73.67 | 57.80 | 9.98 | 0.96 |
| A1 | 1.0 | Spinel | 360 | 6 | 1.89 | 55.46 | 39.64 | 3.88 | 0.59 |
| A1 | 1.0 | Spinel | 370 | 6 | 1.35 | 72.27 | 58.30 | 9.36 | 0.94 |

TABLE 5

| Catalyst Precursor | | | Temperature | WHSV | Me Gain | EB Conversion | Xylene Conversion | Ring Loss |
|---|---|---|---|---|---|---|---|---|
| Example No. | Rh (%) | Support | (° C.) | (hr^{-l}) | (%) | (%) | (%) | (%) |
| A3 | 0.5 | silica, arginine | 350 | 5 | −0.46 | 27.74 | 3.55 | 1.37 |
| A3 | 0.5 | silica, arginine | 350 | 2.5 | −0.81 | 35.24 | 5.12 | 2.08 |
| A3 | 0.5 | silica, arginine | 380 | 2.5 | 0 | 59.42 | 8.79 | 0.17 |
| A3 | 1.0 | silica, arginine | 350 | 5 | −1.89 | 43.8 | 7.04 | 2.49 |
| A3 | 1.0 | silica, arginine | 350 | 2.5 | −2.53 | 55.29 | 9.54 | 3.15 |
| A3 | 1.0 | silica, arginine | 360 | 2.5 | −1.4 | 62.33 | 10.13 | 1.85 |
| A3 | 1.0 | silica, arginine | 370 | 2.5 | −1.65 | 71.32 | 12.23 | 1.24 |
| A4 | 0.5 | silica, ammonia | 350 | 5 | 0.18 | 36.10 | 3.31 | 1.17 |
| A4 | 0.5 | silica, ammonia | 350 | 2.5 | −0.34 | 52.28 | 5.32 | 2.36 |
| A4 | 0.5 | silica, ammonia | 350 | 2.5 | 0.06 | 61.84 | 6.48 | 1.79 |
| A6 (Comp) | 1.0 | silica | 350 | 5 | −0.61 | 2.3 | 0.5 | 0.7 |
| A2 (Comp) | 0.5 | alumina | 350 | 5 | −4.64 | 66.09 | 13.08 | 3.01 |
| A2 (Comp) | 0.5 | alumina | 350 | 5 | −4.17 | 67.96 | 14.34 | 2.46 |
| A2 (Comp) | 0.5 | alumina | 350 | 2.5 | −5.82 | 63.36 | 14.46 | 6.77 |
| A2 (Comp) | 0.5 | alumina | 350 | 2.5 | −5.7 | 72.84 | 16.65 | 4.47 |

TABLE 6

| Catalyst Precursor | | | Temperature | WHSV | Me Gain | Et Conversion | Pr Conversion | TMB Conversion | Ring Loss |
|---|---|---|---|---|---|---|---|---|---|
| Example No. | Rh (%) | Support | (° C.) | (hr^{-l}) | (%) | (%) | (%) | (%) | (%) |
| A2 (Comp) | 1.0 | Alumina | 340 | 4 | −3.62 | 39.81 | 35.87 | 12.35 | 4.35 |
| A2 (Comp) | 1.0 | Alumina | 360 | 4 | −2.11 | 59.35 | 49.83 | 18.06 | 2.00 |
| A2 (Comp) | 1.0 | Alumina | 370 | 4 | −3.23 | 71.07 | 61.92 | 23.88 | 1.48 |
| A2 (Comp) | 1.0 | Alumina | 370 | 6 | −0.59 | 54.49 | 45.06 | 13.85 | 1.06 |
| A5 | 0.5 | Silica | 320 | 3 | −3.26 | 22.97 | 20.64 | 3.77 | 2.32 |
| A5 | 0.5 | Silica | 340 | 3 | −0.39 | 32.40 | 22.28 | 3.70 | 0.89 |
| A5 | 0.5 | Silica | 360 | 3 | 1.27 | 48.53 | 38.27 | 6.06 | 0.84 |
| A5 | 0.5 | Silica | 360 | 2 | 2.39 | 54.31 | 49.23 | 7.25 | 0.97 |
| A5 | 0.5 | Silica | 370 | 2 | 1.98 | 65.15 | 61.95 | 10.75 | 1.38 |
| A5 | 0.5 | Silica | 370 | 2 | 2.09 | 61.70 | 58.53 | 9.47 | 1.14 |
| A5 | 0.5 | Silica | 380 | 3 | 1.61 | 57.99 | 56.74 | 9.15 | 1.02 |
| A5 | 1.0 | Silica | 340 | 4 | −0.10 | 8.71 | 11.90 | 1.31 | 0.74 |
| A5 | 1.0 | Silica | 350 | 2 | 0.36 | 23.30 | 22.33 | 3.81 | 0.74 |
| A5 | 1.0 | Silica | 370 | 2 | 0.77 | 42.92 | 36.98 | 8.51 | 0.52 |
| A5 | 1.0 | Silica | 380 | 2 | 0.29 | 52.43 | 48.04 | 12.07 | 0.27 |
| A5 | 1.0 | Silica | 340 | 3 | 0.74 | 21.04 | 21.00 | 2.07 | 0.89 |
| A5 | 1.0 | Silica | 350 | 2 | 1.40 | 38.66 | 26.51 | 5.21 | 0.74 |
| A5 | 1.0 | Silica | 360 | 2 | 1.99 | 48.70 | 43.72 | 6.68 | 0.55 |
| A5 | 1.0 | Silica | 360 | 1.5 | 1.72 | 57.40 | 52.43 | 9.39 | 0.77 |
| A5 | 1.0 | Silica | 370 | 1.5 | 0.11 | 68.32 | 65.23 | 14.64 | 1.09 |
| A5 | 1.0 | Silica | 360 | 1.5 | 1.96 | 51.97 | 48.83 | 7.41 | 0.77 |

The present disclosure further relates to the following non-limiting embodiments:

A1. A catalyst composition for selective alkyl-demethylation of a C2+-hydrocarbyl-substituted aromatic hydrocarbon, the catalyst composition comprising:

an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and combinations and mixtures of two or more thereof; and

a transition metal element dispersed upon the oxide support material;

wherein the catalyst composition exhibits a hydrogen chemisorption value of at least 15%.

A2. The catalyst composition of A1, wherein the oxide support material exhibits an alpha value of no greater than 2.5, preferably no greater than 2.0, more preferably no greater than 1.5.

A3. The catalyst composition of A1 or A2, wherein the oxide support material is substantially free of a zeolite and alumina.

A4. The catalyst composition of any one of A1 to A3, wherein the oxide support material is selected from the group consisting of CaO, MgO, SrO, silica, a composite oxide of MgO and Al₂O₃, and combinations and mixtures of two or more thereof.

A5. The catalyst composition of any one of A1 to A4, wherein the transition metal element is present in a substantially elemental state.

A6. The catalyst composition of any one of A1 to A5, wherein the transition metal element is selected from the group consisting of Fe, Co, Ni, Ru, Rh, Pd, Re, Os, Ir, Pt, and combinations and mixtures of two or more thereof.

A7. The catalyst composition of any one of A1 to A5, wherein the transition metal element is selected from the group consisting of Rh, Pd, Ir, Pt, and combinations and mixtures of two or more thereof.

A8. The catalyst composition of any one of A1 to A7, wherein the transition metal element is Rh.

A9. The catalyst composition of any one of A1 to A8, wherein the catalyst composition comprises 0.01 to 5 wt % transition metal element, expressed as a weight percentage of the transition metal element in an elemental state relative to a total weight of the catalyst composition.

A10. The catalyst composition of any one of A1 to A8, wherein the catalyst composition comprises 0.1 to 2 wt % transition metal element, expressed as a weight percentage of the transition metal element in an elemental state relative to a total weight of the catalyst composition.

A11. The catalyst composition of any one of A1 to A10, wherein the catalyst composition exhibits a hydrogen chemisorption value of at least 50%.

A12. The catalyst composition of any one of A1 to A11, wherein the oxide support material exhibits a BET surface area of at least 25 m²/g.

B1. A process for converting a C2+-hydrocarbyl-substituted aromatic hydrocarbon, the process comprising:

providing a C6+ aromatic hydrocarbon-containing stream comprising the C2+-hydrocarbyl-substituted aromatic hydrocarbon, wherein the C2+-hydrocarbyl-substituted aromatic hydrocarbon has (i) a C2+ alkyl substitute attached to an aromatic ring therein and/or (ii) an aliphatic ring annelated to an aromatic ring therein; and

contacting the C6+ aromatic hydrocarbon-containing stream with the catalyst composition of any one of A1 to A12 in an alkyl-demethylation zone under alkyl-demethylation conditions effective to convert at least a portion of the C2+-hydrocarbyl-substituted aromatic hydrocarbon to an alkyl-demethylated aromatic hydrocarbon comprising at least one methyl substitute to obtain a first alkyl-demethylated effluent exiting the alkyl-demethylation zone.

B2. The process of B1, wherein contacting exhibits at least one of the following:

a positive methyl gain;

a C2+-alkyl group conversion in a range from 30% to 100%; and

an aromatic ring loss of no greater than 3%.

B3. The process of B1 or B2, wherein the C6+ aromatic hydrocarbon-containing stream comprises at least 80 wt % C8 aromatic hydrocarbons, and at least 5 wt % ethylbenzene based on a total weight of the C6+ aromatic hydrocarbon-containing stream.

B4. The process of B3 , wherein the alkyl-demethylation conditions comprise at least one of the following:

a presence of molecular hydrogen in the alkyl-demethylation zone at a partial pressure of hydrogen in a range from 50 to 2,500 kilopascal absolute;

a temperature in a range from 180 to 500° C.;

an absolute total pressure in a range from 100 to 5,000 kilopascal;

a WHSV in a range from 0.1 to 20 hour⁻¹; and

a molar ratio of molecular hydrogen to the C6+ aromatic hydrocarbon-containing stream in a range from 0.1 to 10.

B5. The process of B4, wherein the C2+-hydrocarbyl-substituted aromatic hydrocarbons in the C6+ aromatic hydrocarbon-containing stream are present substantially in vapor phase in the alkyl-demethylation zone.

B6. The process of any one of B1, B3, B4, and B5, wherein contacting exhibits at least one of the following:
a xylene loss in a range from 0 to 15%;
an ethylbenzene conversion in a range from 30% to 100%;
a positive methyl gain; and
an aromatic ring loss no greater than 3%.

B7. The process of B1 or B2, wherein the C6+ aromatic hydrocarbon-containing stream comprises at least 80 wt % C9+ aromatic hydrocarbons, and at least 20 wt % C2+-hydrocarbyl-substituted C9+ aromatic hydrocarbons, based on a total weight of the C6+ aromatic hydrocarbon-containing stream.

B8. The process of B7, wherein the alkyl-demethylation conditions comprise at least one of the following:

a presence of molecular hydrogen in the alkyl-demethylation zone at a partial pressure of hydrogen in a range from 50 to 2,500 kilopascal absolute;

a temperature in a range from 180 to 500° C.;

an absolute total pressure in a range from 100 to 5,000 kilopascal;

a WHSV in a range from 0.1 to 20 hour⁻¹; and

a molar ratio of molecular hydrogen to the C6+ aromatic hydrocarbon-containing stream in a range from 0.1 to 10.

B9. The process of B8, wherein the C2+-hydrocarbyl-substituted aromatic hydrocarbons in the C6+ aromatic hydrocarbon-containing stream are present substantially in vapor phase in the alkyl-demethylation zone.

B10: The process of any one of B7 to B9, wherein contacting exhibits at least one of the following:

a trimethylbenzenes loss in a range from 0 to 20%;

a C2+-alkyl-substituted C9+ aromatic hydrocarbons conversion in a range from 30% to 100%;

a positive methyl gain; and

an aromatic ring loss no greater than 3%.

B11: The process of B10, wherein contacting further exhibits at least one of the following:
an ethyl group conversion from 30% to 100%; and
a C3-alkyl group conversion from 30% to 100%.

B12: The process of any one of B1 to B11, wherein the alkyl-demethylation catalyst composition is prepared by the process of any one of C1 to C22.

C1. A process for making a catalyst composition for alkyl-demethylation of a C2+-hydrocarbyl-substituted aromatic hydrocarbon, the process comprising:

providing an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and mixtures and combinations of two or more thereof;

providing a source material of a transition metal element;

dispersing the source material of the transition metal element on the oxide support material to obtain a catalyst composition precursor; and

contacting the catalyst composition precursor with a reducing atmosphere under activating conditions to obtain the catalyst composition.

C2. The process of C1, wherein the oxide support material has an alpha value no greater than 2.5, preferably no greater than 2.0, more preferably no greater than 1.5.

C3. The process of C1 or C2, wherein the oxide support material is substantially free of a zeolite and alumina.

C4. The process of any one of C1 to C3, wherein providing an oxide support material comprises:

providing an oxide support precursor; and

contacting the oxide support precursor with an alkaline solution to obtain the oxide support material, wherein the oxide support material comprises surface hydroxyl groups.

C5. The process of C4, wherein the alkaline solution comprises ammonium hydroxide, NaOH, KOH, or a mixture of two or more thereof.

C6. The process of C4 or C5, wherein the oxide support comprises silica.

C7. The process of any one of C1 to C6, wherein the source material of the transition metal element comprises a salt of the transition metal element.

C8. The process of any one of C1 to C7, wherein dispersing the source material of the transition metal element comprises:

forming a liquid dispersion comprising the source material of the transition metal element and a liquid dispersant;

contacting the oxide support material with the liquid dispersion of the source material of the transition metal element to obtain a mixture of the oxide support material and the source material; and

drying the mixture of the oxide support material and the source material to form a dried mixture of the oxide support material and the source material.

C9. The process of C8, wherein the liquid dispersant comprises water.

C10. The process of C8 or C9, wherein the liquid dispersion further comprises a linking agent comprising at least two functional groups capable of promoting linkage between the source material of the transition metal element and the oxide support material.

C11. The process of C10, wherein the linking agent comprises an amino alcohol, an amino acid, a glycol, or a mixture or combination of two or more thereof.

C12. The process of any one of C8 to C11, wherein dispersing the source material of the transition metal element further comprises:

calcining the dried mixture of the oxide support material and the source material at a temperature in a range from 100 to 600° C.

C13. The process of any one of C8 to C11, wherein dispersing the source material of the transition metal element does not include calcining the dried mixture of the oxide support material and the source material.

C14. The process of any one of C1 to C13, wherein after contacting the catalyst composition precursor with a reducing atmosphere under activating conditions, the metal oxide support material exhibits an alpha value of no greater than 2.5, preferably no greater than 2.0, more preferably no greater than 1.5.

C15. The process of any one of C1 to C14, wherein the transition metal element is present substantially in an elemental state after contacting the catalyst composition precursor with the reducing atmosphere under activating conditions.

C16. The process of any one of C1 to C15, wherein the transition metal element is selected from the group consisting of Fe, Co, Ni, Ru, Rh, Pd, Re, Os, Ir, Pt, and combinations and mixtures of two or more thereof.

C17. The process of any one of C1 to C15, wherein the transition metal element is selected from the group consisting of Rh, Pd, Ir, Pt, and combinations and mixtures of two or more thereof.

C18. The process of any one of C1 to C17, wherein the transition metal element is Rh.

C19. The process of any one of C1 to C18, wherein after contacting the catalyst composition precursor with the reducing atmosphere under activating conditions, the catalyst composition comprises 0.01 to 5 wt % transition metal element, expressed as a weight percentage of the transition metal element in an elemental state relative to a total weight of the catalyst composition.

C20. The process of any one of C1 to C18, wherein after contacting the catalyst composition precursor with the reducing atmosphere under activating conditions, the catalyst composition comprises 0.1 to 2 wt % transition metal element, expressed as a weight percentage of the transition metal element in an elemental state relative to a total weight of the catalyst composition.

C21. The process of any of C1 to C20, wherein after contacting the catalyst composition precursor with the reducing atmosphere under activating conditions, the catalyst composition exhibits a hydrogen chemisorption value of at least 15%, preferably at least 50%.

C22. The process of any one of C1 to C21, wherein the activating conditions comprise at least one of the following:

a temperature in a range from 200 to 600° C.;

a hydrogen partial pressure in a range from 5 to 200 kilopascal; and

a total pressure in a range from 100 to 1,000 kilopascal.

D1. A catalyst composition for alkyl-demethylation of a C2+-hydrocarbyl-substituted aromatic hydrocarbon prepared by the process of any one of C1 to C22.

E1. A process for converting a C2+-hydrocarbyl-substituted aromatic hydrocarbon, the process comprising:

providing a C6+ aromatic hydrocarbon-containing stream comprising the C2+-hydrocarbyl-substituted aromatic hydrocarbon, wherein the C2+-hydrocarbyl-substituted aromatic hydrocarbon has (i) a C2+alkyl substitute attached to an aromatic ring therein and/or (ii) an aliphatic ring annelated to an aromatic ring therein; and

contacting the C6+ aromatic hydrocarbon-containing stream with the catalyst composition of D1 in an alkyl-demethylation zone under alkyl-demethylation conditions effective to convert at least a portion of the C2+-hydrocarbyl-substituted aromatic hydrocarbon to an alkyl-demethylated aromatic hydrocarbon comprising at least one methyl substitute, optionally to obtain a first alkyl-demethylated effluent exiting the alkyl-demethylation zone.

F1. A catalyst composition precursor prepared in the process of any one of C1 to C22.

F2. The catalyst composition precursor of F1, comprising the oxide support material, the source material of the transition metal element, and the linking agent.

F3. The catalyst composition of F1 or F2, wherein the oxide support material comprises surface hydroxyl groups.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments are presented herein. Not all features of a physical implementation are described or shown in this application for the sake of clarity. It is understood that in the development of a physical embodiment incorporating the features of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps. Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A catalyst composition for selective alkyl-demethylation of a C2+-hydrocarbyl-substituted aromatic hydrocarbon, the catalyst composition comprising:
an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and combinations and mixtures of two or more thereof; and
a transition metal element dispersed upon the oxide support material;
wherein the catalyst composition exhibits a hydrogen chemisorption value of at least 15%.

2. The catalyst composition of claim 1, wherein the oxide support material exhibits an alpha value of no greater than 2.5.

3. The catalyst composition of claim 1, wherein the oxide support material is substantially free of a zeolite and alumina.

4. The catalyst composition of claim 1, wherein the oxide support material is selected from the group consisting of CaO, MgO, SrO, silica, a composite of MgO and Al₂O₃, and combinations and mixtures of two or more thereof.

5. The catalyst composition of claim 1, wherein the transition metal element is present in a substantially elemental state.

6. The catalyst composition of claim 1, wherein the transition metal element is selected from the group consisting of Fe, Co, Ni, Ru, Rh, Pd, Re, Os, Ir, Pt, and combinations and mixtures of two or more thereof.

7. The catalyst composition of claim 1, wherein the transition metal element is selected from the group consisting of Rh, Pd, Ir, Pt, and combinations and mixtures of two or more thereof.

8. The catalyst composition of claim 1, wherein the transition metal element is Rh.

9. The catalyst composition of claim 1, wherein the catalyst composition comprises 0.01 to 5 wt % transition metal element, expressed as a weight percentage of the transition metal element in an elemental state relative to a total weight of the catalyst composition.

10. The catalyst composition of claims, wherein the catalyst composition comprises 0.1 to 2 wt % transition metal element, expressed as a weight percentage of the transition metal element in an elemental state relative to a total weight of the catalyst composition.

11. The catalyst composition of claim 1, wherein the catalyst composition exhibits a hydrogen chemisorption value of at least 50%.

12. The catalyst composition of claim 1, wherein the oxide support material exhibits a BET surface area of at least 25 m²/g.

13. A process for converting a C2+-hydrocarbyl-substituted aromatic hydrocarbon, the process comprising:
providing a C6+ aromatic hydrocarbon-containing stream comprising the C2+-hydrocarbyl-substituted aromatic hydrocarbon, wherein the C2+-hydrocarbyl-substituted aromatic hydrocarbon has (i) a C2+alkyl substitute attached to an aromatic ring therein and/or (ii) an aliphatic ring annelated to an aromatic ring therein; and
contacting the C6+ aromatic hydrocarbon-containing stream with the catalyst composition of any one of claims 1-12 in an alkyl-demethylation zone under alkyl-demethylation conditions effective to convert at least a portion of the C2+-hydrocarbyl-substituted aromatic hydrocarbon to an alkyl-demethylated aromatic hydrocarbon comprising at least one methyl substitute to obtain a first alkyl-demethylated effluent exiting the alkyl-demethylation zone.

14. The process of claim 13, wherein contacting exhibits at least one of the following:
a positive methyl gain;
a C2+-alkyl group conversion in a range from 30% to 100%; and
an aromatic ring loss of no greater than 3%.

15. The process of claim 13, wherein the C6+ aromatic hydrocarbon-containing stream comprises at least 80 wt % C8 aromatic hydrocarbons, and at least 5 wt % ethylbenzene based on a total weight of the C6+ aromatic hydrocarbon-containing stream.

16. The process of claim 15, wherein the alkyl-demethylation conditions comprise at least one of the following:
a presence of molecular hydrogen in the alkyl-demethylation zone at a partial pressure of hydrogen in a range from 50 to 2,500 kilopascal absolute;
a temperature in a range from 180 to 500° C.;
an absolute total pressure in a range from 100 to 5,000 kilopascal;
a WHSV in a range from 0.1 to 20 hour⁻¹; and
a molar ratio of molecular hydrogen to the C6+ aromatic hydrocarbon-containing stream in a range from 0.1 to 10.

17. The process of claim 16, wherein the C2+-hydrocarbyl-substituted aromatic hydrocarbons in the C6+ aromatic hydrocarbon-containing stream are present substantially in vapor phase in the alkyl-demethylation zone.

18. The process of claim 13, wherein contacting exhibits at least one of the following:
a xylene loss in a range from 0% to 15%;
an ethylbenzene conversion in a range from 30% to 100%;
a positive methyl gain; and
an aromatic ring loss no greater than 3%.

19. The process of claim 13, wherein the C6+ aromatic hydrocarbon-containing stream comprises at least 80 wt % C9+ aromatic hydrocarbons, and at least 20 wt % C2+-hydrocarbyl-substituted C9+ aromatic hydrocarbons, based on a total weight of the C6+ aromatic hydrocarbon-containing stream.

20. The process of claim 19, wherein the alkyl-demethylation conditions comprise at least one of the following:
a presence of molecular hydrogen in the alkyl-demethylation zone at a partial pressure of hydrogen in a range from 50 to 2,500 kilopascal absolute;
a temperature in a range from 180 to 500° C.;
an absolute total pressure in a range from 100 to 5,000 kilopascal;
a WHSV in a range from 0.1 to 20 hour⁻¹; and
a molar ratio of molecular hydrogen to the C6+ aromatic hydrocarbon-containing stream in a range from 0.1 to 10.

21. The process of claim 20, wherein the C2+-hydrocarbyl-substituted aromatic hydrocarbons in the C6+ aromatic hydrocarbon-containing stream are present substantially in vapor phase in the alkyl-demethylation zone.

22. The process of claim 19, wherein contacting exhibits at least one of the following:
a trimethylbenzenes loss in a range from 0% to 20%;
a C2+-alkyl-substituted C9+ aromatic hydrocarbons conversion in a range from 30% to 100%;
a positive methyl gain; and
an aromatic ring loss no greater than 3%.

23. The process of claim 22, wherein contacting further exhibits at least one of the following:
an ethyl group conversion from 30% to 100%; and
a C3-alkyl group conversion from 30% to 100%.

24. The process of claim 13, wherein the catalyst composition is prepared by a process comprising:
providing an oxide support material selected from the group consisting of an alkaline earth metal oxide, silica, a composite of an alkaline earth metal oxide and Al₂O₃, a composite of ZnO and Al₂O₃, a lanthanide oxide, a composite of a lanthanide oxide and Al₂O₃, and mixtures and combinations of two or more thereof;
providing a source material of a transition metal element;
dispersing the source material of the transition metal element on the oxide support material to obtain a catalyst composition precursor; and
contacting the catalyst composition precursor with a reducing atmosphere under activating conditions to obtain the catalyst composition.
